(19) United States
(12) Patent Application Publication
(10) Pub. No.: US 2022/0306768 A1
(43) Pub. Date: Sep. 29, 2022

(54) REDIRECTING DEATH-INDUCING SIGNAL COMPLEX (DISC) BY MODIFYING DEATH RECEPTOR AGONIST TO INDUCE CELL DEATH FOR CANCER TREATMENT

(71) Applicant: CITY OF HOPE, Duarte, CA (US)

(72) Inventor: Edward Wenge WANG, Azusa, CA (US)

(73) Assignee: CITY OF HOPE, Duarte, CA (US)

(21) Appl. No.: 17/703,523

(22) Filed: Mar. 24, 2022

Related U.S. Application Data
(60) Provisional application No. 63/165,514, filed on Mar. 24, 2021.

(51) Int. Cl. C07K 19/00; C07K 14/47; C07K 14/715; A61P 35/00

(52) U.S. Cl. CPC C07K 19/00; C07K 14/4747; C07K 14/7151; A61P 35/00; A61K 38/00

(57) ABSTRACT

It was found that TRAIL-induced death signaling was largely diminished by lysosomal degradation system. Inhibition of lysosomal degradation by small molecules led to accumulation of DR5 in lysosomes and reversed TRAIL resistance in almost all cancer cells. Redirecting TRAIL-induced signaling away from lysosomal degradation may therefore induce massive cell death and overcome TRAIL resistance. Taking advantage of bioactive protein transduction domains (PTD) and signaling peptides, such as TAT peptide from HIV TAT protein, NLS (nuclear localization signal), MTS (mitochondrial targeting sequence), a series of new TRAILs fused with these peptides was constructed. It was found that TRAIL fused with bioactive peptide exerted remarkably high potency in inducing cell death in cancer cells, but not normal cells.

### PRIORITY CLAIM

This application claims the benefit of U.S. Provisional Patent Application No. 63/165,514, filed Mar. 24, 2021, which is incorporated by reference herein in its entirety.

### STATEMENT REGARDING FEDERALLY SPONSORED RESEARCH

This invention was made with government support under Grant Number K12CA001727, awarded by the National Institutes of Health. The government has certain rights in the invention.

### SEQUENCE LISTING

This application contains a Sequence Listing, which was submitted in ASCII format via EFS-Web, and is hereby incorporated by reference in its entirety. The ASCII copy, created on Mar. 18, 2022, is named SequenceListing.txt and is 7 KB in size.

### BACKGROUND

Inducing selective cell death in cancer cells is the ultimate goal of pharmaceutical research for cancer treatment. Cancer cell death can be induced through two distinct pathways, intrinsic and extrinsic apoptotic pathways. Almost all current cancer chemotherapeutics trigger cell death by activating intrinsic pathways that involve mitochondria. On the other hand, the well characterized extrinsic pathway via death receptors expressed on the cell surface has not yet been successfully targeted for cancer treatment. It has been demonstrated that death receptors, including DR4 and DR5, once triggered by a ligand termed TRAIL (tumor necrosis factor-related apoptosis-inducing ligand), can selectively pass death signaling from outside of the cell surface into cells, inducing apoptosis in cancer cells but not affecting normal cells (Twomey et al., 2015).

Preclinical studies have shown promising anti-cancer effects of TRAIL in various cancer models. Clinically, phase I studies showed that recombinant human TRAIL is well tolerated (Herbst et al., 2010); however, in phase II and phase III studies, TRAIL—in combination with standard chemotherapy regimens in various cancer types—failed to realize the significant clinical outcomes expected based on the earlier models. (Twomey et al., 2015; Quintavalle and Condorelli, 2012; Ouyang et al., 2018). The discrepancy between preclinical and clinical outcomes can be explained by TRAIL resistance of primary tumor cells (Ashkenazi, 2015). Thus, developing death receptor agonists that overcome TRAIL resistance is a desired strategy for producing more efficient cancer therapeutics.

### SUMMARY

In certain embodiments, a fusion protein comprising the tumor necrosis factor-related apoptosis-inducing ligand (TRAIL) fused to (i) a cell penetrating peptide (CPP) or (ii) a signal peptide is provided. In some embodiments, the CPP or signal peptide component of the fusion protein include a TAT peptide (TAT), a nuclear localization signal (NLS), a mitochondrial targeting sequence (MTS), or a hemagglutinin (HA) peptide.

In other embodiments, an expression construct comprising a fusion gene inserted into an expression vector is provided, the fusion gene comprising a nucleotide sequence encoding the TRAIL fused in frame with a nucleotide sequence encoding a cell penetrating peptide (CPP) or a signal peptide. In some embodiments, the CPP or signal peptide component of the fusion protein is a TAT peptide (TAT) from HIV TAT protein, a nuclear localization signal (NLS), a mitochondrial targeting sequence (MTS), or a hemagglutinin (HA) peptide.

In certain embodiments, the fusion protein and/or the expression constructs described herein, alone or as part of a pharmaceutical composition, can be used in methods of killing cancer cells or treating cancers.

### BRIEF DESCRIPTION OF THE DRAWINGS

This application contains at least one drawing executed in color. Copies of this application with color drawing(s) will be provided by the Office upon request and payment of the necessary fees.

FIG. 1 is a schematic showing the effect of certain therapies on the death-inducing signaling complex according to certain embodiments.

FIG. 2 is a schematic showing the effect of small molecules on the death-inducing signaling complex according to certain embodiments.

FIG. 3A shows death receptor accumulation in lysosome, whereby inhibition of lysosomal degradation by small molecules leads to accumulation of DR5 in lysosomes and reverses TRAIL resistance in almost all cancer cells according to certain embodiments.

FIG. 3B shows lysosomotropic small molecule treated cells compared to control according to certain embodiments.

FIG. 3C is a gel showing accumulated levels of DR5 induced by small molecules according to certain embodiments.

FIG. 4 is a schematic showing strategies of redirecting the death-inducing signaling complex through multiple target peptides according to certain embodiments.

FIG. 5 shows that TAT-TRAIL induces faster and massive cell death in a TRAIL-sensitive cancer cell line, compared with the His-tagged parental TRAIL construct according to certain embodiments.

FIG. 6 shows in vivo testing of TAT-TRAIL using ovarian cancer xenograft models, OVCAR3 cells and A2780 cells, implanted intraperitoneally according to certain embodiments. Preliminary data show notable antitumor effects.

FIG. 7 shows eight new TRAIL constructs using different variations of CCPs or in combination fused with a His-tag (for purification) according to certain embodiments.

FIG. 8 shows that TAT-TRAIL induces higher caspase activity as compared to control and His-tagged parental TRAIL in TRAIL-sensitive cancer cell lines. Of the 60 NCI cancer cell lines, 33 TRAIL-sensitive cells lines become more sensitive to TAT-TRAIL, while 17 of TRAIL-resistant cell lines become sensitive to TAT-TRAIL, only 10 cell lines remain resistant.

FIG. 9 shows cell viability following administration of TAT-TRAIL compared to His-tagged parental TRAIL in the Hep3B cell line according to certain embodiments. After 48 hours, the IC50 of TAT-TRAIL is low, demonstrating approximately sixty times greater potency as compared to the His-tagged parental TRAIL.

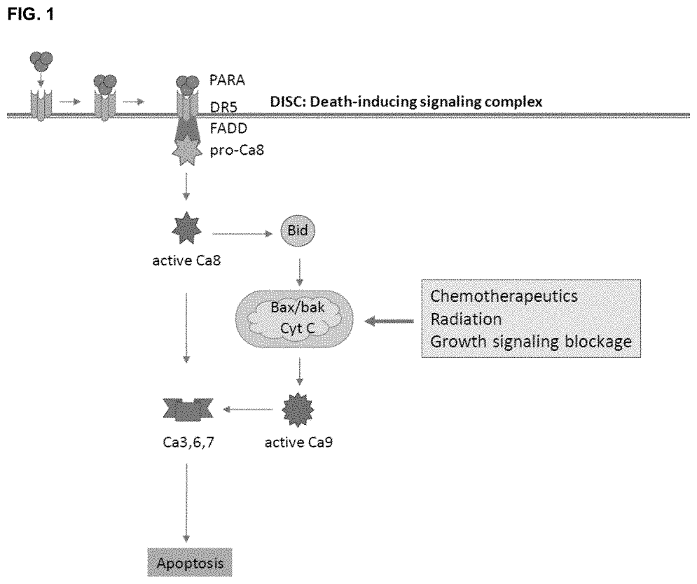

### DETAILED DESCRIPTION

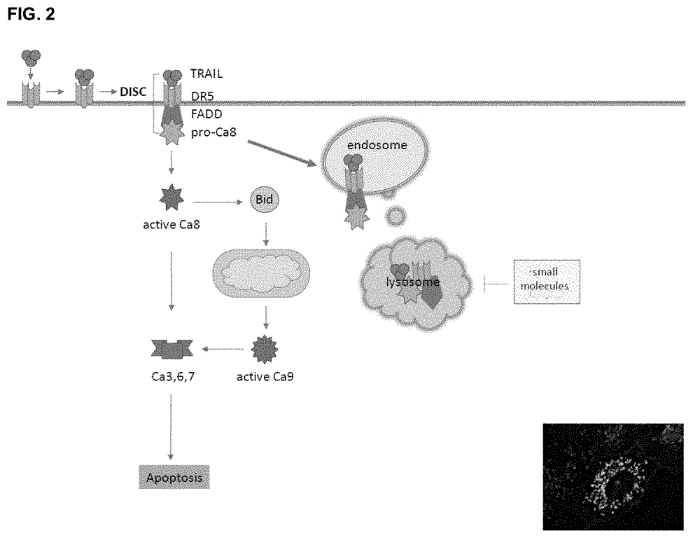

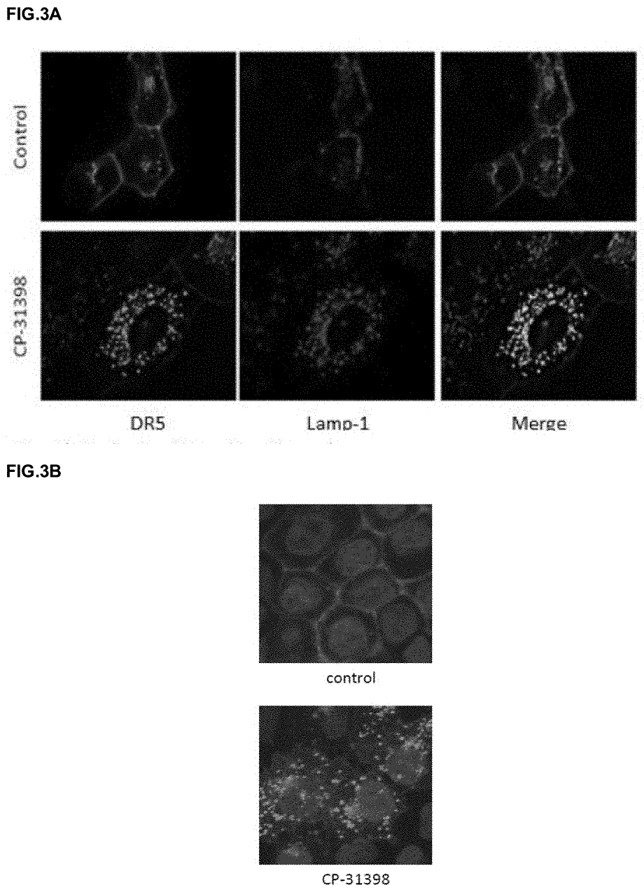

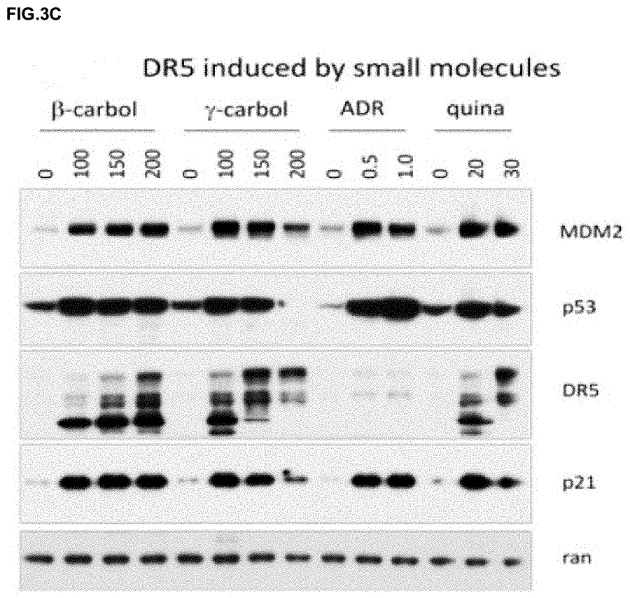

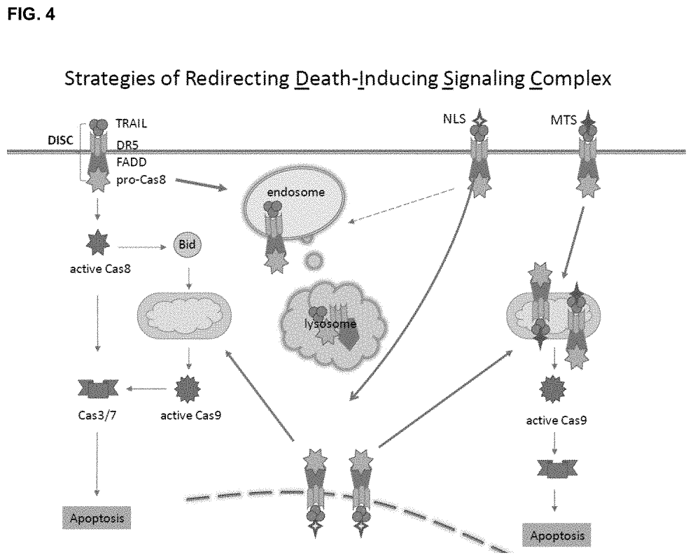

TRAIL-induced death signaling is largely diminished by the lysosomal degradation system. Inhibition of lysosomal degradation by small molecules leads to accumulation of DR5 in lysosomes and reverses TRAIL resistance in almost all cancer cells, as shown in FIGS. 2, 3A-3C [6]. To redirect TRAIL-induced signaling away from lysosomal degradation and overcome TRAIL resistance (see FIG. 4), fusion proteins to target cell surface death receptors and methods for their use are provided herein.

According to the embodiments described below, the fusion proteins include tumor necrosis factor-related apoptosis-inducing ligand (TRAIL) component fused to one or more delivery moieties that evades lysosomal degradation. In certain embodiments, the delivery moiety or moieties are fused to the N-terminus of the TRAIL. In other embodiments, the TRAIL component is connected or fused to the one or more delivery moieties via a linker.

The TRAIL component may be any form of TRAIL suitable for targeting, binding, and/or activating death receptors on a tumor cell (e.g., including DR4 and DR5). For example, the TRAIL component of the fusion proteins disclosed herein may be the native TRAIL protein (e.g., Homo sapiens TNF superfamily member 10 (TNFSF10) transcript variant 1 (hTRAIL)) or a portion thereof; see NCBI Reference Sequence NM_003810), a recombinant TRAIL protein (e.g., dulanermin) or a portion thereof, a synthetic TRAIL protein or a portion thereof, a codon optimized TRAIL protein, or a modified TRAIL protein or portion thereof that includes one or more mutations as compared to a native TRAIL. In one embodiment, the TRAIL component comprises amino acids 94-281 of hTRAIL (hTRAIL₉₄₋₂₈₁). The amino acid sequence of hTRAIL is shown below (hTRAIL₉₄₋₂₈₁ is underlined):

hTRAIL:
(SEQ ID NO: 1)
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGT [symbol] GRSNTLSSPNSKNEKALGRK
INSWESSRSGHSFLSNLHLRNGELVIHEKGFYYIYSQTYFRFQEEIKENT
KNDKQMVQYIYK [symbol] T [symbol] YPDPILLMKSAR [symbol] SCWSKDAEYGLYSIYQGG [symbol]
FELKENDRIFVSVTNEHLIDMDHEASFFGAFLVG

In one embodiment, the TRAIL is a modified TRAIL protein that includes one or more mutations in each monomeric unit. In certain embodiments, the one or more mutations occur at amino acid residues 130, 213, 215, 228, 247 (in bold italics, above), or any combination thereof. In certain embodiments the modified TRAIL protein includes one or more of the following mutations: a substitution of G in place of R at residue 130 (R130G), a substitution of Win place of Y at residue 213 (Y213W), a substitution of D in place of S at residue 215 (S215D), a substitution of S in place of N at residue 228 (S228N), a substitution of V in place of I at residue 247 (I247V).

Delivery moieties that may be fused to the TRAIL component for use in the embodiments described herein may include, but are not limited to, a cell penetrating peptide (CPP) (CPPs are also referred to herein as a protein transduction domain (PTD)) and/or a signal peptide that can cross plasma membranes and/or direct the fusion protein within a cell's cytoplasm to the nucleus, an organelle, or other subcellular compartment. Nonlimiting examples of such delivery moieties that may be used to design the fusion proteins described herein may include a protein derived CPP derived from a natural protein (e.g., peptides or fragments derived from HIV Tat transactivator protein, Penetratin, pVEC, VP22 peptide, Bac7, Pyrrhocoricin, Cotamine, Melittin, human lactoferrin, bLFcin, ARF(1-22)), a chimeric CPP partly derived from two or more segments from different proteins or peptides (e.g., transportan, TP10) and which may contain a signal sequence, a synthetic CPP (e.g., synthetic peptides, oligoarginines, peptide nucleic acids (PNAs)), a nuclear localization signal or sequence (NLS), or a mitochondria targeting sequence (MTS). Specific delivery moieties that may be fused to TRAIL to design fusion proteins in accordance with the embodiments described herein may be found in the following, which are hereby incorporated by reference as if fully disclosed herein: Durzyńska et al., Viral and other cell-penetrating peptides as vectors of therapeutic agents in medicine, J Pharmacol Exp Ther 354:32-42, July 2015; Borrelli et al., Cell penetrating peptides as molecular carriers for anti-cancer agents, Molecules 2018, 23, 295; CPPsite 2.0 Database of Cell-Penetrating Peptides, see https://webs.iiitd.edu.in/raghava/cppsite/information.php). In some embodiments, the fusion protein may include one delivery moieties, while in other embodiments, the fusion protein may include more than one delivery moiety.

In certain embodiments the fusion protein includes a TRAIL component fused to a TAT peptide (TAT), which may be referred to herein as a TAT-TRAIL fusion protein or just TAT-TRAIL. In some embodiments, the TAT peptide comprises an amino acid sequence of RKKRRQRRR (SEQ ID NO:2).

In certain embodiments, the fusion protein includes a TRAIL fused to a nuclear localization signal (NLS).

In certain embodiments, the fusion protein includes a TRAIL fused to a mitochondria targeting sequence (MTS).

In certain embodiments, the fusion proteins described herein may also include a targeting moiety to increase specificity of delivery of the fusion protein to tumor cells. For example, in one embodiment, the fusion protein may include a homing peptide or active targeting ligand that bind targets highly expressed on tumor cells. Examples of targeting moieties that may be included in the fusion proteins described herein include, but are not limited to, hem agglutinin (HA), and RGD peptides.

In certain embodiments, the fusion proteins described herein may also include one or more protein tags to assist with the expression and/or purification of the fusion proteins. Protein tags are small peptides used to improve a protein's stability, solubility, affinity, purification, or a combination thereof. Suitable protein tags that may be used in the fusion protein include, but are not limited to, FLAG, hemaglutinin antigen, polyhistidine (His), HBH, myc, S-tag, V5, glutathione S-transferase (GST), small ubiquitin-related modifier (SUMO), maltose-binding protein (MBP), Thioredoxin A (TRX)), tandem affinity purification (TAP), and calmodulin binding peptide (CBP). In some embodiments, the fusion proteins include a poly-His tag, a SUMO tag, or both.

In accordance with the embodiments described above, the fusion proteins described herein have a sequence that includes components arranged according to one or more of the schematic arrangements shown below (n 1):

5′-(protein tag)ₙ-(delivery moiety)ₙ-(TRAIL
component)-3′
5′-(protein tag)ₙ-(delivery moiety)ₙ-(targeting
moiety)-(TRAIL cornponent)-3′
5′-(protein tag)ₙ-(delivery moiety)ₙ-(targeting
moiety)-(delivery moiety)ₙ-(TRAIL component)-3′

Accordingly, in certain embodiments, the components and corresponding amino acid sequences of a fusion protein may be arranged as follows:

5′-poly-His-NLS-TRAIL component-3′
5′-poly-His-NLS-HA-TRAIL component-3′
5′-poly-His- [symbol] -TRAIL component-3′
5′-poly-His- [symbol] -HA-TRAIL component-3′
5′-poly-His-SUMO- [symbol] -TRAIL component-3′
5′-poly-His-MTS-TRAIL component-3′
5′-poly-His-MTS-HA-TRAIL component-3′
5′-poly-His- [symbol] -MTS-TRAIL component-3′
5′-poly-His- [symbol] -HA-MTS-TRAIL component-3′
5′-poly-His- [symbol] -NLS-TRAIL component-3′
5′-poly-His- [symbol] -HA-NLS-TRAIL component-3′
5′-poly-His- [symbol] -HA-NLS-MTS-TRAIL component-3′

The schematic arrangements above represent templates for order of the components, regardless of the sequences used. In certain embodiments, additional sequences (e.g., a linker) may be present in between the components shown above to account for spacers, linkers, hinge regions, etc.

In one embodiment, the components and corresponding amino acid sequence of a TAT-TRAIL fusion protein may be arranged as follows:

5′-poly-His- [symbol] -hTRAIL₉₄₋₂₈₁-3′
(SEQ ID NO: 3)
5′-HHHHHH- [symbol] -
RTSEETISTVQEKQQNISPLVRERGPQRVAAHITGTRGRS
NTLSSPNSKNEKALGRKINSWESSRSGHSFLSNLHLRNGELVIHEKGFYY
IYSQTYFRFQEEIKENTKNDKQMVQYIYKYTSYPDPILLMKSARNSCWSK
DAEYGLYSIYQGGIFELKENDRIFVSVTNEHLIDMDHEASFFGAFLVG-
3′

In another embodiment, the components and corresponding amino acid sequence of another TAT-TRAIL fusion protein may be arranged as follows:

5′-poly-His-SUMO- [symbol] -hTRAIL₉₄₋₂₈₁-3′
(SEQ ID NO: 4)
5′-HHHHHH-XXXXXXXXXXXXXXXXXXX- [symbol] -
RTSEETISTVQEKQQNISP
LVRERGPQRVAAHITGTRGRSNTLSSPNSKNEKALGRKINSWESSRSGHS
FLSNLHLRNGELVIHEKGFYYIYSQTYFRFQEEIKENTKNDKQMVQYIYK
YTSYPDPILLMKSARNSCWSKDAEYGLYSIYQGGIFELKENDRIFVSVTN
EHLIDMDHEASFFGAFLVG-3′

In some embodiments, the TAT-TRAIL fusion protein includes a modified TRAIL protein that includes one or more mutations in the TRAIL component. For example, in such embodiments, the one or more mutations occur at amino acid residues 130, 213, 215, 228, 247 or any combination thereof, as discussed above. In certain embodiments, the modified TRAIL protein includes one or more of the following mutations: a substitution of G in place of R at residue 130 (R130G), a substitution of Win place of Y at residue 213 (Y213W), a substitution of D in place of S at residue 215 (S215D), a substitution of S in place of N at residue 228 (S228N), a substitution of V in place of I at residue 247 (I247V).

In some embodiments, the TAT-TRAIL fusion protein includes a TRAIL protein sequence of various lengths, for example, TAT-TRAIL (95-281), TAT-TRAIL (114-281).

### Expression of the Fusion Protein

To express a fusion protein described above, an expression cassette is provided. The expression cassette includes a nucleotide sequence that encodes the fusion protein, also referred to herein as “fusion nucleotide sequence” or “fusion nucleotide molecule”. Such a nucleotide may include a nucleotide sequence corresponding to any sequence of degenerate codons that give rise to the desired amino acid sequence in accordance with the genetic code.

In certain embodiments the fusion nucleotide sequences that encode the fusion proteins described herein have a nucleotide sequence that corresponds to the component arrangements described above. For example, in one embodiment, the components and corresponding nucleotide sequence of the 5′-poly-His-TAT-hTRAIL₉₄₋₂₈₁-3′ fusion protein above may be provided according to the genetic code.

In another embodiment, the components and corresponding nucleotide sequence of the 5′-poly-His-SUMO-TAT-hTRAIL₉₄₋₂₈₁-3′ fusion protein above may be provided according to the genetic code.

In one embodiment, the CPP (or PTD) is a 9-amino acid TAT peptide that is fused to the N-terminal of TRAIL. The fusion protein is further fused to a tag at its N-terminal. In the preferred embodiment, the tag is a His tag. In other embodiments, the tag may be FLAG, Myc, V5, or any other suitable tag known in the art.

The fusion protein described herein may form a TRAIL monomer, a TRAIL dimer, or a TRAIL trimer.

In one embodiment, the TRAIL is fused to a Fc domain at the C-terminal. In another embodiment, the TRAIL is fused a Trimer-Tag at the C-terminal. In the preferred embodiment, TRAIL is fused to Trimer-Tag to form a more stable covalently-linked trimer (Liu 2017).

### Expression of the TRAIL Fusion Gene

According to the embodiments described herein, a fusion protein expression system is provided herein. In one embodiment, the fusion gene may be inserted into a vector for delivery to target cells. Any suitable vector may be used, for example, a bacterial vector, a viral vector, or a eukaryotic expression vector. In the preferred embodiment, the vector is a bacterial plasmid vector.

In some embodiments, a gene encoding the fusion protein is provided, wherein the gene may include a nucleotide sequence encoding a TRAIL that is fused in frame to a nucleotide sequence encoding a CPP (or PTD) or a signal peptide, and a nucleotide sequence encoding a small ubiquitin-related modified (SUMO) protein.

In some embodiments, the nucleotide sequence encoding a TRAIL is a codon-optimized TRAIL sequence. In these embodiments, a nucleotide sequence encoding a His-tag and a nucleotide sequence encoding a small ubiquitin-related modified (SUMO) protein are used to stabilize the expressed protein to achieve optimal expression.

In one embodiment, the nucleotide sequence encoding the CPP (or PTD) may encode but is not limited to a TAT peptide (TAT) or a hemagglutinin (HA). In another embodiment, the nucleotide sequence encoding the signal peptide may encode but is not limited to a nuclear localization signal (NLS) or a mitochondrial targeting sequence (MTS).

In some embodiments, the nucleotide sequence encoding the CPP (or PTD) or signal peptide is a 9-amino acid TAT peptide that is fused in frame to the 5′ end of the nucleotide sequence encoding TRAIL, and the nucleotide sequence encoding the SUMO protein is fused in frame to the 5′ end of the nucleotide or nucleic acid sequence encoding the CPP (or PTD) or signal peptide. In a certain embodiment, the nucleotide sequence encoding a tag is fused to the 5′ end of the nucleotide sequence encoding the SUMO protein. In the preferred embodiment, the tag is a His tag. In other embodiments, the tag may be FLAG, Myc, V5, or any other suitable tag known in the art.

In some embodiments, the nucleotide sequence encoding the TRAIL encodes a modified TRAIL protein that includes one or more mutations as compared to native TRAIL.

In one embodiment, the nucleotide sequence encoding the TRAIL is fused in frame to a nucleotide sequence encoding an Fc domain. In another embodiment, the nucleotide sequence encoding the TRAIL is fused in frame to a nucleotide sequence encoding a Trimer-Tag.

In certain embodiments, the nucleotide sequence encoding the Fc domain or the Trimer-tag is fused in frame to the 3′ end of the nucleotide sequence encoding the TRAIL.

Treatment of Cancer with TAT-TRAIL

This TRAIL fusion can be used to treat any suitable cancer cell, including but not limited to bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, and uterine cancer. In addition, the methods may be used to treat tumors that are malignant (e.g., cancers) or benign (e.g., hyperplasia, cyst, pseudocyst, hamartoma, and benign neoplasm).

In certain embodiments, ovarian cancer (e.g., ovarian cancer cells, ovarian cancer tumor) is used in initial studies as a model to develop new TRAILs and provides a target cancer for development of new TRAILs due to the following reasons. First, ovarian cancer typically presents at a later stage, with one third of patients with malignant ascites when initially diagnosed. It is easy to access primary ovarian cancer cells for laboratory testing. Second, ovarian cancer is usually restricted within the abdomen and pelvis, and local delivery of therapeutics has been established and proved more effective than systemic treatment. Third, current treatment of ovarian cancer is primarily limited to surgery and chemotherapy, and new treatments are urgently needed to help patients suffering from this deadly disease.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

A “therapeutically effective amount,” “effective amount” or “effective dose” is an amount of a composition (e.g., a therapeutic agent) that produces a desired therapeutic effect in a subject, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, Pa., 2005.

The cancer cells used in the methods described above may be from an in vitro culture system, or they may exist as part of a tumor (e.g., a primary tumor, a metastatic tumor, a malignant tumor or a benign tumor). When the cells are part of a tumor, the composition may be administered in an effective amount to a subject having cancer.

Also disclosed herein are pharmaceutical compositions comprising a fusion protein as described herein and one or more suitable carriers or excipients. Examples of suitable carriers may include physiologically acceptable solutions, such as sterile saline and buffered saline. The excipient can be a natural or synthetic substance, and can act as a filler or diluents for the at least one nucleic acid, facilitating administration to the subject. The excipient can also facilitate nucleic acid uptake into a target cell, or otherwise enhance the effectiveness of the targeting molecule.

In some embodiments, the pharmaceutical composition may include a pharmaceutically effective amount of an adjuvant. Any immunologic adjuvant that may stimulate the immune system and increase the response to a vaccine or pharmaceutical composition, without having any specific antigenic effect itself may be used as the adjuvant. Many immunologic adjuvants mimic evolutionarily conserved molecules known as pathogen-associated molecular patterns (PAMPs) and are recognized by a set of immune receptors known as Toll-like Receptors (TLRs). Examples of adjuvants that may be used in accordance with the embodiments described herein include Alum, Freund's complete adjuvant, Freund's incomplete adjuvant, double stranded RNA (a TLR3 ligand), LPS, LPS analogs such as monophosphoryl lipid A (MPL) (a TLR4 ligand), flagellin (a TLR5 ligand), lipoproteins, lipopeptides, single stranded RNA, single stranded DNA, imidazoquinolin analogs (TLR7 and TLR8 ligands), CpG DNA (a TLR9 ligand), Ribi's adjuvant (monophosphoryl-lipid A/trehalose dicorynoycolate), glycolipids (α-GalCer analogs), unmethylated CpG islands, oil emulsion, liposomes, virosomes, saponins (active fractions of saponin such as QS21), muramyl dipeptide, alum, aluminum hydroxide, squalene, BCG, cytokines such as GM-CSF and IL-12, chemokines such as MIP 1-α and RANTES, activating cell surface ligands such as CD40L, N-acetylmuramine-L-alanyl-D-isoglutamine (MDP), and thymosin α1. The amount of adjuvant used can be suitably selected according to the degree of symptoms, such as softening of the skin, pain, erythema, fever, headache, and muscular pain, which might be expressed as part of the immune response in humans or animals after the administration of this type of vaccine.

In further embodiments, use of various other adjuvants, drugs or additives with the pharmaceutical composition of the invention, as discussed above, may enhance the therapeutic effect achieved by the administration of the pharmaceutical composition. The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); antibiotics; and PEG.

In some embodiments, pharmaceutical composition described herein may be used in combination with other known pharmaceutical products, and may further comprise other drugs and additives. Examples of drugs or additives that may be used in conjunction with a pharmaceutical composition described herein include drugs that aid intracellular uptake of the composition disclosed herein, liposome and other drugs and/or additives that facilitate transfection, (e.g., fluorocarbon emulsifiers, cochleates, tubules, golden particles, biodegradable microspheres, and cationic polymers).

In some embodiments, use of various other adjuvants, drugs or additives with the pharmaceutical composition of the invention, as discussed above, may enhance the therapeutic effect achieved by the administration of the pharmaceutical composition.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The pharmaceutical composition that may be used in accordance with the methods described herein may be administered, by any suitable route of administration, alone or as part of a pharmaceutical composition. A “route of administration” may refer to any administration pathway known in the art, including but not limited to aerosol, enteral, nasal, ophthalmic, oral, parenteral, rectal, transdermal (e.g., topical cream or ointment, patch), or vaginal. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to and should not be construed to limit its scope in any way. The examples do not include detailed descriptions of conventional methods. Such methods are well known to those of ordinary skill in the art and are described in numerous publications. Further, all references cited above and in the examples below are hereby incorporated by reference in their entirety, as if fully set forth herein.

### EXAMPLES

### Example 1: Development of a New Generation of TRAIL for Treating Cancer

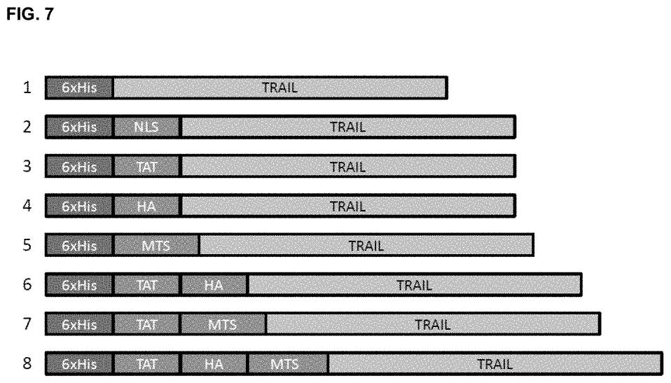

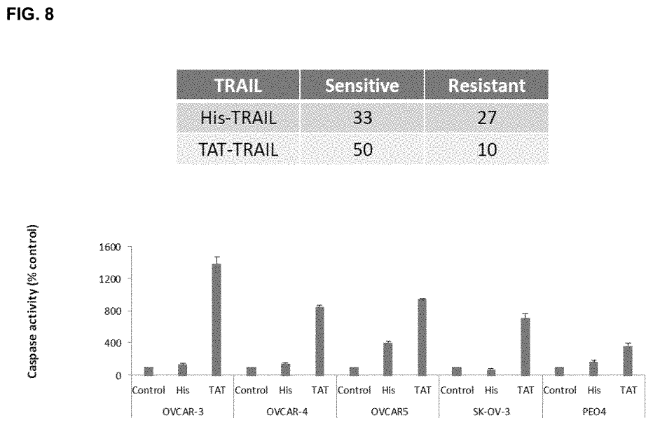

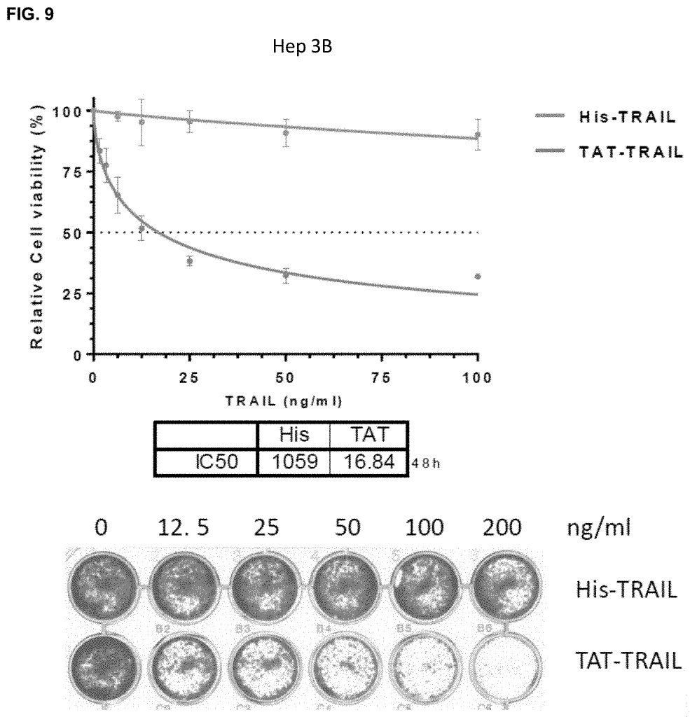

Redirecting TRAIL-induced signaling away from lysosomal degradation induced massive cell death and overcame TRAIL resistance. This was done by fusing bioactive protein transduction domains (PTD—or CPPs) and signaling peptides, such as TAT peptide from HIV TAT protein, NLS (nuclear localization signal), MTS (mitochondrial targeting sequence), with TRAIL to construct a series of new TRAILs, as shown in FIG. 7. TRAIL fused with a 9-amino acid, bioactive TAT peptide (RKKRRQRRR, SEQ ID NO:2), exerted remarkable high potency in inducing cell death in cancer cells but not normal cells, as shown in FIGS. 8 and 9. Testing of this TAT-TRAIL was done on cancer cell lines and not only induced quick cell death in a much shorter time (less than 1 hour), but also induced massive cell death in TRAIL-resistant cancer cells, as shown in FIG. 9. The results demonstrate that TRAIL fused with signaling peptides redirects TRAIL-induced signaling away from lysosomal degradation and overcomes TRAIL resistance.

### Example 2: Anti-Tumor Efficacy of TAT-TRAIL In Vitro

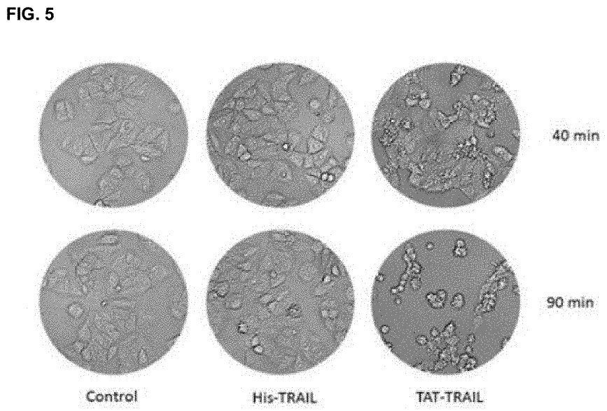

TAT-TRAIL was tested in an NCI 60-cancer cell panel, which included 60 different human cancer cell lines set up by the National Cancer Institute for cancer therapeutics screening. Among the 60 cell lines of various cancer types, 33 were sensitive to parental TRAIL, but 27 were resistant. TAT-TRAIL induced faster and massive cell death in all 33 TRAIL-sensitive cell lines, compared with the His-tagged parental TRAIL construct, as shown in FIG. 5. Further, TAT-TRAIL executed effective cell death in 17 out of the 27 cancer cell lines that were resistant to parental TRAIL, with a potency increased by up to a hundred times. The results demonstrate that TAT-TRAIL induced faster and more efficient tumor cell death in vitro.

To account for any differences in TRAIL sensitivity between cell lines and primary cancer cells, TAT-TRAIL may be tested on primary ovarian cancer cells obtained from malignant ascites of ovarian cancer patients.

### Example 3: Anti-Tumor Efficacy of TAT-TRAIL In Vivo

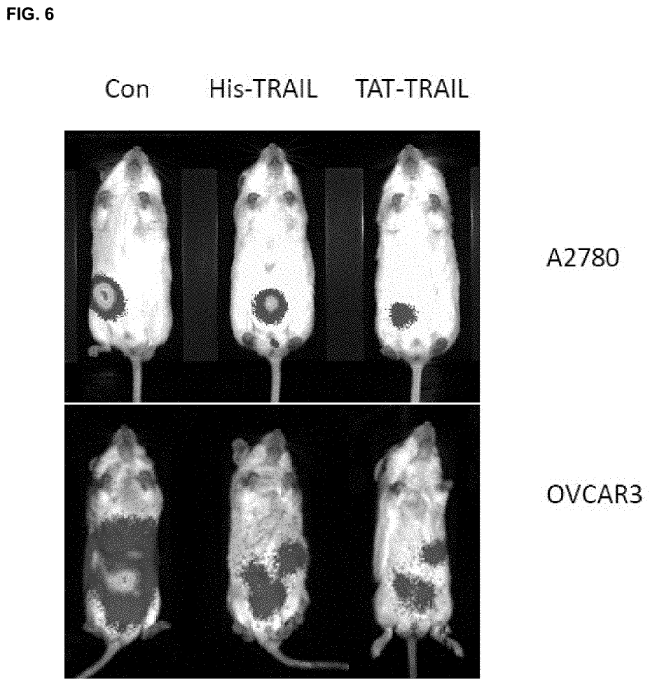

In vivo testing was performed using ovarian cancer xenograft models, OVCAR3 and A2780, implanted intraperitoneally. Preliminary data showed remarkable antitumor effects, as shown in FIG. 6. The results demonstrate that TAT-TRAIL induced faster and more efficient tumor cell death in vivo.

Tests of TAT-TRAIL may also be done on the PDX model of ovarian cancer. The administration protocol may be optimized to guide future clinical trial design for ovarian cancer treatment protocols in clinic trails with the goal that the TAT-TRAIL protocol may be used for treatment of other types of cancer, as TRAIL-induced cancer cell death is not restricted to any specific tissue type.

From the foregoing, it will be appreciated that specific embodiments of the invention have been described herein for purposes of illustration, but that various modifications may be made without deviating from the scope of the invention. Accordingly, the invention is not limited except as by the appended claims.

1. A fusion protein comprising a tumor necrosis factor-related apoptosis-inducing ligand (TRAIL) fused to (i) a cell penetrating peptide (CPP) or (ii) a signal peptide.

2. The fusion protein of claim 1, wherein the CPP or signal peptide is selected from TAT peptide (TAT), nuclear localization signal (NLS), mitochondrial targeting sequence (MTS), or hemagglutinin (HA).

3. The fusion protein of claim 2, wherein the CPP or signal peptide is a TAT peptide having an amino acid sequence comprising RKKRRQRRR.

4. The fusion protein of claim 1, wherein the CPP or signal peptide is fused to the N-terminal of TRAIL.

5. The fusion protein of claim 4, wherein the fusion protein is further fused to a His-tag at its N-terminal.

6. The fusion protein of claim 1, wherein the TRAIL is a TRAIL monomer, a TRAIL dimer, or a TRAIL trimer.

7. The fusion protein of claim 1, wherein the TRAIL is a modified TRAIL protein comprising one or more mutations in each monomeric unit.

8. The fusion protein of claim 1, wherein the TRAIL is fused to an Fc domain or a Trimer-Tag.

9. (canceled)

10. The fusion protein of claim 8, wherein the Fc domain or Trimer-Tag is fused to the C-terminal of TRAIL.

11. An expression construct comprising a fusion gene inserted into an expression vector, the fusion gene comprising a nucleotide sequence encoding a TRAIL fused in frame to
(i) a nucleotide sequence encoding a cell penetrating peptide (CPP) or a signal peptide; and
(ii) a nucleotide sequence encoding a small ubiquitin-related modifier (SUMO) protein.

12. The expression construct of claim 11, wherein the nucleotide sequence encoding a TRAIL comprises a codon-optimized TRAIL sequence.

13. The expression construct of claim 11, wherein the nucleotide sequence encoding the CPP or signal peptide encodes a TAT peptide (TAT), a nuclear localization signal (NLS), a mitochondrial targeting sequence (MTS), or a hemagglutinin d (HA).

14. The expression construct of claim 13, wherein the nucleotide sequence encoding the CPP or signal peptide is a TAT peptide having an amino acid sequence comprising RKKRRQRRR.

15. The expression construct of claim 11, wherein the (i) nucleotide sequence encoding the CPP or signal peptide is fused in frame to the 5′ end of the nucleotide sequence encoding the TRAIL.

16. The fusion protein of claim 11, wherein the nucleotide sequence encoding the TRAIL encodes a modified TRAIL protein comprising one or more mutations as compared to native TRAIL.

17. The expression construct of claim 11, wherein the nucleotide sequence encoding the TRAIL is fused in frame to a nucleotide sequence encoding an Fc domain or a Trimer-Tag.

18. (canceled)

19. The expression construct of claim 17, wherein the nucleotide sequence encoding the Fc domain or the Trimer-Tag is fused in frame to the 3′ end of the nucleotide sequence encoding the TRAIL.

20. The expression construct of claim 11, wherein the expression vector is a plasmid.

21. A method of killing a cancer cell comprising contacting the cancer cell with an effective dose of a fusion protein comprising a tumor necrosis factor-related apoptosis-inducing ligand (TRAIL) fused to (i) a cell penetrating peptide (CPP) or (ii) a signal peptide.

22. The method of claim 21, wherein the cancer cell is an ovarian cancer cell.

23-27. (canceled)


SEQUENCE LISTING (4 sequences)

SEQ ID NO: 1 - protein, 281 aa, Artificial Sequence [sequence omitted]

SEQ ID NO: 2 - protein, 9 aa, Artificial Sequence
  TAT peptide
RKKRRQRRR

SEQ ID NO: 3 - protein, 203 aa, Artificial Sequence
  TAT-TRAIL
HHHHHHRKKRRQRRRRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 4 - protein, 222 aa, Artificial Sequence
  TAT-TRAIL
  misc_feature (7)..(25): Xaa can be any naturally occurring amino acid
HHHHHHXXXXXXXXXXXXXXXXXXXRKKRRQRRRRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG